(19) United States
(12) Patent Application Publication
(10) Pub. No.: US 2021/0379277 A1
(43) Pub. Date: Dec. 9, 2021

(54) PERSONAL HYGIENE DEVICE

(71) Applicant: All-Klean, Inc., Los Angeles, CA (US)

(72) Inventors: Martin Lee Hopp, Los Angeles, CA (US); David Michael Morelock, Escondido, CA (US)

(73) Assignee: All-Klean, Inc., Los Angeles, CA (US)

(21) Appl. No.: 17/341,148

(22) Filed: Jun. 7, 2021

Related U.S. Application Data
(60) Provisional application No. 63/036,242, filed on Jun. 8, 2020.

(51) Int. Cl. A61M 3/02; A61L 2/10; A61L 2/26; B08B 3/02

(52) U.S. Cl. CPC A61M 3/0258; A61M 3/0279; A61L 2/10; A61L 2/26; E03D 9/085; A61L 2202/21; A61L 2202/11; A61L 2202/17; B08B 3/026

(57) ABSTRACT

A hand-held personal hygiene device includes a main body having an upper end, a lower end, and a pump therein. The pump is configured to pump water or another fluid from an inlet port at the upper end to an outlet port at the lower end. A reservoir has a lower base configured to removably seat in the upper end of the main body to engage and open a self-closing valve with the inlet port of the main body. A tubular nozzle member has a lower end comprising one or more nozzle outlets and an upper end configured to be removably secured to the outlet port of the main body causing liquid pumped by the pump to flow through the tubular nozzle member to the one or more nozzle outlets. The reservoir and the main body may be aligned to form a liquid flow path that may be held vertically to allow gravity flow from the reservoir to the pump.

### CROSS-REFERENCE TO RELATED APPLICATIONS

This application claims the benefit of Provisional No. 63/036,242 (Attorney Docket No. 55533-703.101), filed Jun. 8, 2020, the entire content of which is incorporated herein by reference.

### BACKGROUND OF THE INVENTION

### 1. Field of the Invention

The present invention relates generally to the field of medical devices and methods. More particularly, the present invention relates to handheld personal hygiene devices and methods of their use.

The need for personal hygiene can be increased under various circumstances, such as child birth, surgeries on urinary, genital and rectal anatomies. Women often suffer from scabbing, crusting, mucous, biological degree, and raw surfaces following child birth. Both women and men require heighted personal hygiene following hemorrhoid and rectal surgery.

Performing hygiene of the urinary, genital and rectal anatomies presents particular challenges, particularly on posterior regions. The posterior anatomies involved are difficult for the patient to directly observe, can be very sensitive, and can be difficult to clean by conventional washing with conventional wash cloths and similar tools.

As an alternative the use of wash cloths, squeeze wash bottles, such as the Frida Mom Upside Down Peri Bottle, commercially available from Fridababy, Miami, Fla., have been developed for use with infants. The Peri Bottle consists of a plastic squeeze bottle and a screw-on applicator tube with a bent tip. Stream velocity, volume, and duration are difficult to control by manually squeezing the bottle, and the plastic bottle and tube are difficult to aim. Moreover, the plastic bottles and their fluid contents are subject to bacterial contamination, presenting an infection risk for the patient. Additionally, it can be difficult to deliver a medicament, cleanser, or other material in the fluid stream. While some materials can be dissolved, suspended or otherwise combined in the bulk fluid in the squeeze bottle, the concentration of some materials can unacceptably vary within the bottle and many materials are incapable of being dissolved or suitably suspended in the fluid.

For these reasons, it would be desirable to provide a personal hygiene product which is an improvement over a plastic squeeze bottle and other presently available personal hygiene products. More specifically, it would be desirable to provide a personal hygiene product which provides precise and continuous control of the stream volume, pressure, and duration, as well as reducing the risk of contamination and enhancing the ability to combine and deliver medicaments, cleansers, and other materials in the fluid stream. At least some of these objectives will be met by the inventions described herein.

### 2. Listing of the Background Art

Relevant patents include U.S. Pat. Nos. 5,097,540; 5,864,895; 6,751,813; 7,155,754; and 7,670,141.

### SUMMARY OF THE INVENTION

In a first aspect, the present invention provides a hand-held personal hygiene device which includes a main body, a pump, a motor, a reservoir, and a tubular nozzle member. The main body typically has an upper end with an inlet port and a lower end with an outlet port. The main body has an interior which is filled with a number of components including a pump and a motor. The pump is configured to pump a fluid from the inlet port of the main body to the outlet port at the lower end of the main body. A motor in the interior of the main body is connected to drive the pump. Typically, the motor is powered by a battery which is also included in the main body, and often the battery is rechargeable as described in greater detail below.

The reservoir has a lower base configured to removably seat in the upper end of the main body and a self-closing valve that engages the inlet port of the main body to open and allow fluid in the reservoir to flow to the pump when the lower base is seated in the upper end of the main body. The tubular nozzle member typically has a lower end comprising one or more nozzle outlets and an upper end configured to be removeably secured to the outlet port of the main body. Fluid pumped by the pump in the main body will flow through the tubular nozzle member to the one or more nozzle outlets when the tubular nozzle member is connected to the lower end of the main body and the pump is operated.

In preferred aspects, the reservoir and the main body are aligned to form a liquid flow path that may be held vertically to allow gravity flow from the reservoir to the pump. This feature allows the hand-held personal hygiene device to be used in a vertical orientation to deliver fluid, typically an aqueous cleaning solution, optionally an aqueous solution containing a medicament or other material substance intended to treat the patient, cleanse the patient, or the like. As will be described in greater detail below, the user may hold the main body in one hand and perform all necessary operational functions using the one hand while typically maintaining the device in a generally vertical orientation.

In still other preferred aspects, the main body may have an upper cylindrical section to fit a user's hand and a lower conical section to connect to the tubular nozzle member. In some instances, the upper cylindrical section and the lower conical section each have a longitudinal axis. In such instances, the axis of the lower conical section may typically be oriented at an angle in a range from 5° to 45°, preferably 10° to 30°, relative to the axis of the upper cylindrical section to help deflect and orient the attachment of the tubular nozzle member. While the reservoir and the main body will typically be held in a generally vertical orientation, it's often desirable to have the tubular nozzle member deviate outwardly from the vertical axis in order to access certain patient anatomies.

In many embodiments, the motor is controlled by an on/off button, capable of pulsed and/or continuous operation of the pump and motor, as well as both a flow increase button and flow decrease button adapted to change the flow rate and/or pressure. In certain instances, the on/off button will be oriented on a first side of the cylindrical section in the main body so that the user can operate the on/off button with either a thumb or with one or more fingers. The flow increase and decrease buttons can then be located on an opposite side of the cylindrical section in the main body so that the user can operate the flow increase and decrease buttons using the other of the thumb and finger(s). To be clear, the user will be operating the on/off button with either a thumb or fingers while operating the flow increase/decrease buttons with either the fingers or the thumb.

In certain embodiments, the reservoir may have at least one screw attachment, press fit attachment, latched attachment, or the like, for connection to the upper end of the main body.

In still other specific instances of the present invention, the self-closing valve of the reservoir comprises a spring-loaded valve that is configured to be displaced upwardly to open and allow fluid flow from the reservoir. Typically, the inlet port of the main body comprises a displacement element positioned to displace a valve plug of the self-closing outlet valve of the reservoir upwardly to open said self-closing valve when the reservoir is attached to the main body.

In still further instances of the hand-held personal hygiene devices of the present invention, a pair of latching members may be disposed at the upper end of the tubular nozzle member. Such latching members engage locking surfaces in an otherwise open lower end of the main body when the upper end of the tubular nozzle member is inserted into the open lower end of the main body. Positioning such latching members on the tubular nozzle member allows the positioning of release buttons in a lower wall of the conical section of the main body, where the release buttons are configured to be pushed inwardly to disengage the latching members from the locking surfaces. In particular, the release buttons may be disposed on opposite sides of the conical section of the main body so that the user can squeeze the release buttons between a thumb and finger to disengage the tubular nozzle member from the conical section of the main body. After disengagement, a new, clean tubular nozzle member can be connected with the main body, optionally having a different geometry or configuration for cleaning different regions of the anatomy.

In still further aspects of the present invention, the personal hygiene device may further include a material release cartridge which can carry any one of a number of materials, including cleansing materials, medicaments, and the like. The material release cartridge will typically be configured to be inserted between the lower end of the reservoir and the upper end of the main body. To allow the personal hygiene device to be used either with or without material release cartridge, the material release cartridge will typically have an upper end which is similar in construction to the upper end of the main body and a lower end which is similar in construction to the lower end of the reservoir. An internal volume of the material release cartridge will be configured to be filled with a soluble or dispersible material having a desired property or properties. In specific instances, the upper end of the material release cartridge will have an inlet port configured to mate with and open the self-closing valve in the bottom of the reservoir and a lower end with a self-closing valve configured to be opened by the inlet port of the main body when the material release cartridge is seated in the upper end of the main body.

In a still further aspect of the present invention, the hand-held personal hygiene device may be configured to disinfect the fluid being delivered to the user. In particular, the device may comprise a disinfecting energy structure in the inlet end of the main body. More specifically, the disinfecting energy structure may comprise one or more light emitting diodes (LEDs) which direct ultraviolet energy into an interior of the reservoir when the reservoir is seated in the upper end of the main body.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a personal hygiene device constructed in accordance with the principles of the present invention.

FIG. 2 is an exploded view of the personal hygiene device of FIG. 1.

FIG. 3 is a detail view of an exemplary upper connection end of a replaceable fluid delivery wand of the personal hygiene device of the present invention.

FIG. 4 is a detail view of an exemplary fluid delivery tip of the replaceable fluid delivery wand of the personal hygiene device of the present invention.

FIG. 5 is an exploded view of a main body of the personal hygiene device of FIG. 1.

FIGS. 6A and 6B illustrate the upper connection end of the replaceable fluid delivery wand of FIG. 3 and a mating connection structure in a lower end of the main body in both a separated configuration (FIG. 6A) and an attached configuration (FIG. 6B).

FIGS. 7A and 7B illustrate an upper connection end of the main body and a mating connection structure in a lower end of a fluid reservoir of the personal hygiene device of the present invention in both a separated configuration (FIG. 7A) and an attached configuration (FIG. 7B).

FIG. 8 illustrates the addition of a diffusing cartridge in personal hygiene device in accordance with the principles of the present invention.

FIG. 9 is a cross-sections view of the diffusing cartridge of FIG. 8.

FIG. 10 illustrates the personal hygiene device in a user's hand in accordance with the principles of the present invention.

FIG. 11 illustrates insertion of the main body of the personal hygiene device of the present invention into a recharging receptacle.

FIG. 12 illustrates the personal hygiene device further including structure for disinfecting fluid while the fluid is maintained within the reservoir.

### DETAILED DESCRIPTION OF THE INVENTION

The illustrations presented herein are not actual views of any particular catheter insertion device but are merely idealized representations employed to describe example embodiments of the present disclosure. The following description provides specific details of embodiments of the present disclosure in order to provide a thorough description thereof. However, a person of ordinary skill in the art will understand that the embodiments of the disclosure may be practiced without employing many such specific details. Indeed, the embodiments of the disclosure may be practiced in conjunction with conventional techniques employed in the industry. In addition, the description provided below does not include all elements to form a complete structure or assembly. Only those process acts and structures necessary to understand the embodiments of the disclosure are described in detail below. Additional conventional acts and structures may be used. Also note, any drawings accompanying the application are for illustrative purposes only and are thus not drawn to scale. Additionally, elements common between figures may have corresponding numerical designations.

As used herein, the terms “comprising,” “including,” and grammatical equivalents thereof are inclusive or open-ended terms that do not exclude additional, un-recited elements or method steps, but also include the more restrictive terms “consisting of,” “consisting essentially of,” and grammatical equivalents thereof.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structure and the apparatus in a predetermined way.

As used herein, the singular forms following “a,” “an,” and “the” are intended to include the plural forms as well, unless the context clearly indicates otherwise.

As used herein, the term “and/or” includes any and all combinations of one or more of the associated listed items.

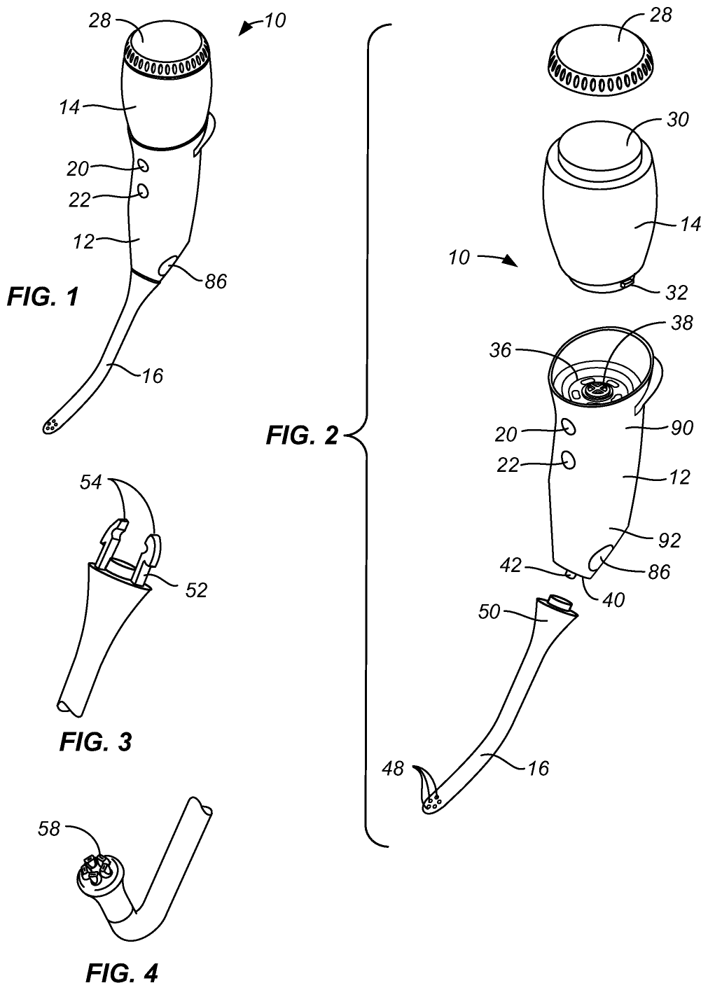

As used herein, spatially relative terms, such as “below,” “lower,” “bottom,” “above,” “upper,” “top,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, the spatially relative terms may refer to a personal hygiene device when the device is disposed in a vertical orientation (e.g., the position depicted in FIG. 1).

As used herein, the term “substantially” in reference to a given parameter, property, or condition means and includes to a degree that one of ordinary skill in the art would understand that the given parameter, property, or condition is met with a degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

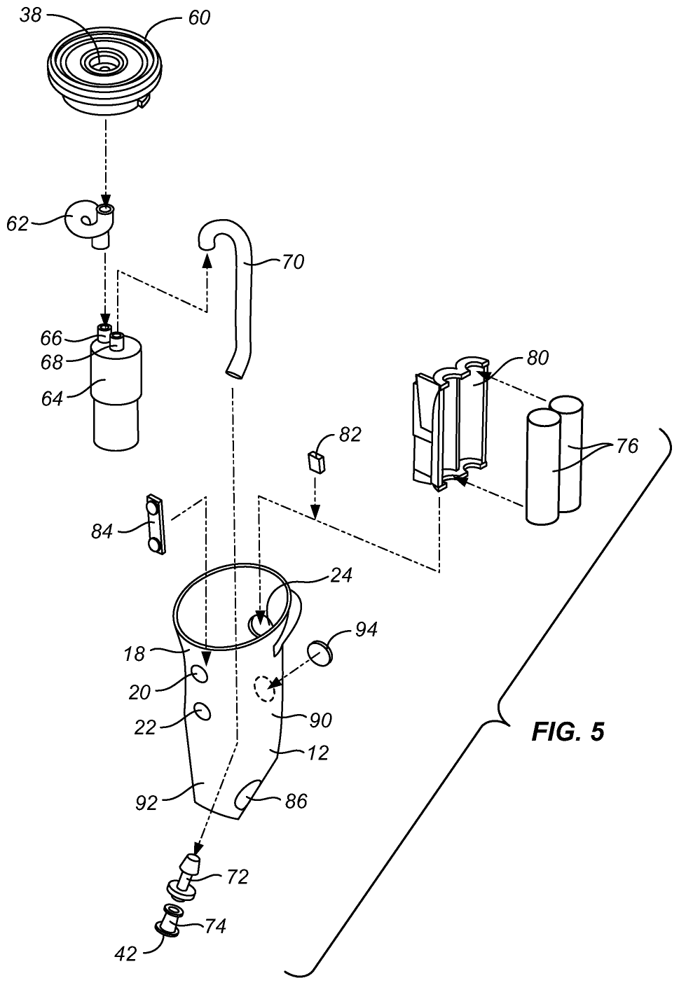

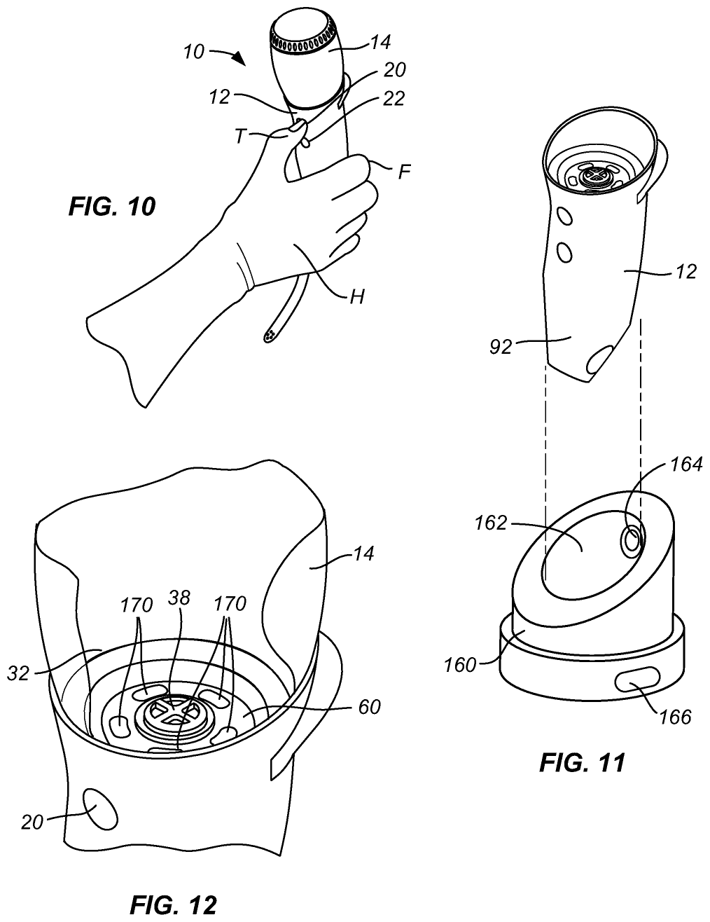

Referring now to FIGS. 1 and 2, a personal hygiene device 10 configured to be held in a user's hand, typically in a single hand, includes a main body 12, a reservoir 14, and a tubular nozzle member 16. A flow increase button 20 and a flow decrease button 22 are vertically aligned on one exterior surface of the main body 12, while an on/off button 24 (as best seen in FIG. 5) is disposed on the opposite surface. As described in more detail below with respect to FIG. 10, such opposed alignment of the control button allows the user to operate both the on/off function and the pressure or flow rate delivery function using a single hand.

The reservoir 14 is typically a clear plastic canister having an open end 30 for filling, a removable lid 28 for covering the open end, and a lower base 32 for removable placement in an upper end 36 of the main body 12. The removable cap 28 is typically screwed on while the lower base 32 may be attached to the upper end 36 of the main body by any conventional means, including screwable threads, bayonet attachments, press-fit attachments, and the like.

The main body 12 has an inlet port 38 and an open receptacle in the upper end 36 and an outlet port 42 at its lower end 40.

The tubular nozzle member 16 has an upper end 50 configured to connect to the lower end 40 of the main body, as will be described in more detail with respect to FIG. 3 hereinbelow, and a lower end 46 having a plurality of outlet nozzles 48.

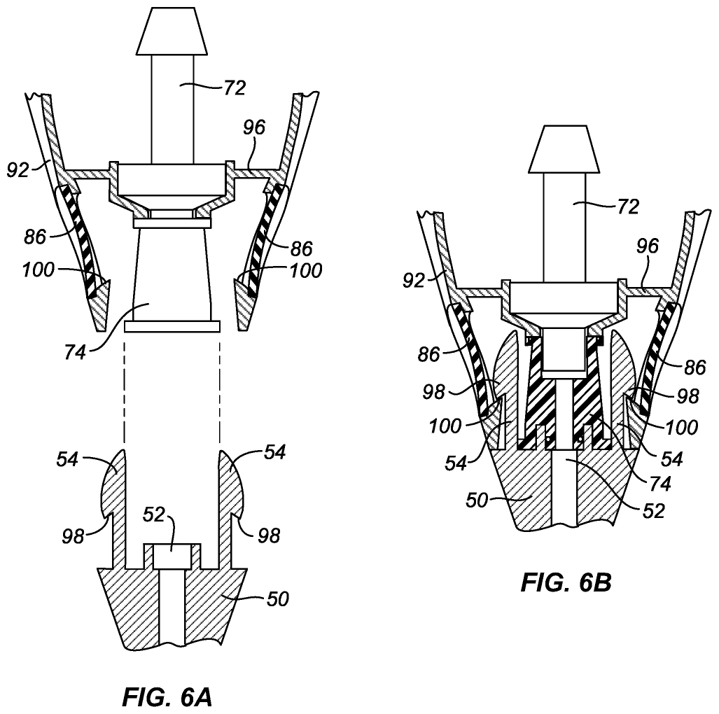

A detailed view of an exemplary upper end 50 of the tubular nozzle member 16 is shown in FIG. 3. An inlet port 52 is configured to mate with the outlet port 42 of the main body 12 when the tubular nozzle member 16 is attached to the main body. Attachment to the main body 12 is preferably achieved using a pair of latching members 54 which may be pressed into the lower end 40 of the main body 12, as described in more detail with respect to FIGS. 6A and 6B below. The lower or outlet end of the tubular nozzle member 16 may take a variety of forms or geometries. For example, as shown in FIG. 4, the outlet end may be bent with a nozzle head 58 at an angle relative to the longitudinal axis of member, typically in the range from 30 degrees to 120 degrees.

Referring now to FIG. 5, the internal components of the main body 12 will be described in greater detail. The main body 12 typically includes an outer housing or shell 18 having an exterior surface and an interior volume. The shell 18 typically has an upper cylindrical section 90 and a lower conical section 92. The longitudinal axis of the upper cylindrical section will typically be oriented at an angle in the range from 5° to 45°, usually from 10° to 30°, relative to a longitudinal axis of the lower conical section 92. In this way, the lower conical section is somewhat bent or deviated relative to the cylindrical section which will typically be held in the user's hand. The on/off button 24 is typically formed on a surface of the upper cylindrical section 90 which is generally opposite to that of the section in which the flow increase/decrease buttons 20 and 22 are formed. A pair of release buttons 86 is also formed through the wall of the lower cylindrical section 90 and will be disposed and configured to disengage the latching members 54 of the tubular nozzle member 16, as described in more detail below with respect to FIGS. 6A and 6B.

An upper body cap 60 includes the inlet port 38 of the main body 12. An inlet tube 62 is connected to a bottom of the inlet port 38 and is disposed to connect to an inlet 66 on a pump unit 64. An outlet tube 70 is similarly configured to connect to an outlet 68 on the pump 64 in order to deliver fluid from the main body inlet 38 via the pump to a check valve 72 which is received in a lower body cap 74. The assembly of the check valve 72 and lower body cap 74 provides the outlet port 42 which connects to the upper end 50 of the tubular nozzle member 16, as described in more detail below with respect to FIGS. 6A and 6B.

Battery 76, which typically are rechargeable, is received in a sled 80 which supports the batteries as well as supporting the pump 64. An on/off printed circuit board (PCB) 82 is provided to engage the on/off button 24 and to correspondingly control a motor of the pump 64. Similarly, a flow/pressure adjustment PCB 84 is provided to couple to the flow increase button 20 and flow decrease button 22 to allow the user to control the motor speed and output of the pump 64. Pump 64 will typically be a centrifugal pump but could be a peristaltic or other positive displacement pump as well.

Referring now to FIGS. 6A and 6B, the attachment and detachment of the tubular nozzle member 16 from the lower end of the main body 12 will be described. FIG. 6A is a partial, cross-sectional view of the lower conical section 92 of the main body 12 showing the check valve 72 and the lower body cap 74. The check valve 72 will be connected to the outlet tube 70 to deliver fluid to the check valve and outwardly through the lower body cap 74.

Latching members 54 on the upper end 50 of the tubular nozzle member 16 may be inserted through a space between the open bottom end of the shell 18 and an outer surface of the lower body cap 74. After being inserted through this annular opening, hooks 98 formed in the latching members 54 will engage locking surfaces 100 in the lower end of the lower conical section 92, as shown in FIG. 6B. The latching members 54 may be disengaged by simply pressing inwardly on the release buttons 86 which may be simple, flexible membranes in openings in the wall of the lower conical section 92. The tubular nozzle member may then be simply pulled out from the lower end of the main body 12, allowing cleaning, replacement, substitution, or the like.

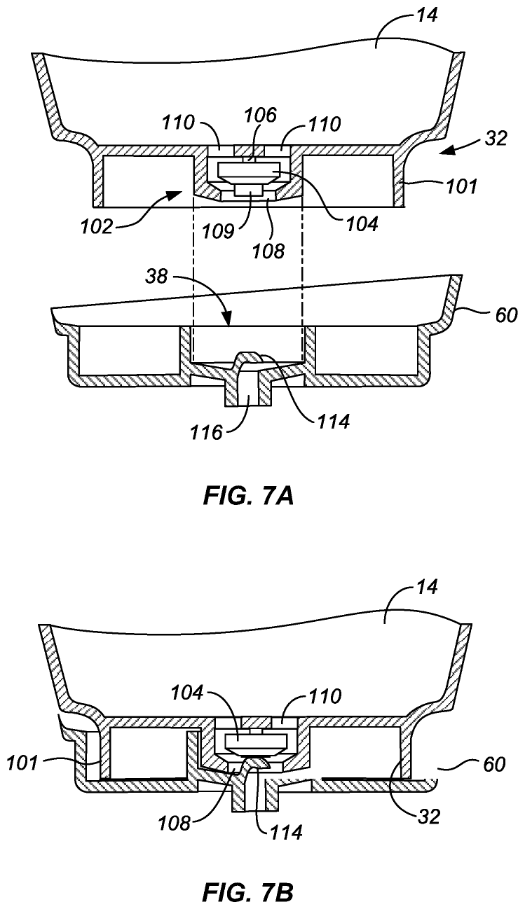

Referring now to FIGS. 7A and 7B, attachment of the reservoir 14 to the upper end of the main body 12 will be described. The lower base 32 of the reservoir 14 has an annealer wall or skirt 101 that surrounds a one-way valve structure 102. The valve structure, in turn, comprises a valve plug 104 having a spring attachment 106. The spring attachment allows the valve plug to be displaced upwardly from seat 108 to open the aperture 109 in the seat to allow flow through the valve. So long as the reservoir is not mounted on the main body, however, the valve structure will remain closed and any fluid in the interior of the reservoir will be prevented from flowing through the valve.

The self-closing valve 104 will automatically open when the reservoir 14 is mounted on the main body 12 as will now be described. The upper body cap 60 of the main body 12 includes the inlet port 38 which has a displacement element 114 disposed over an inlet opening 116. When the lower end of the reservoir 14 is inserted into the upper body cap 60, as shown in FIG. 7B, the displacement member 114 engages a lower surface of the valve plug 104, thus raising the valve plug against the spring force in order to raise the valve plug from over the seat 104. This opens the valve allowing fluid from the reservoir to flow through the opening on 16 and into the inlet tube 70 from where it is pumped as described previously with respect to FIG. 5. As soon as the reservoir 14 is detached from the upper body cap 60, the spring will close the valve plug 104 against the valve seat 108 to close the valve and prevent further fluid flow from the reservoir.

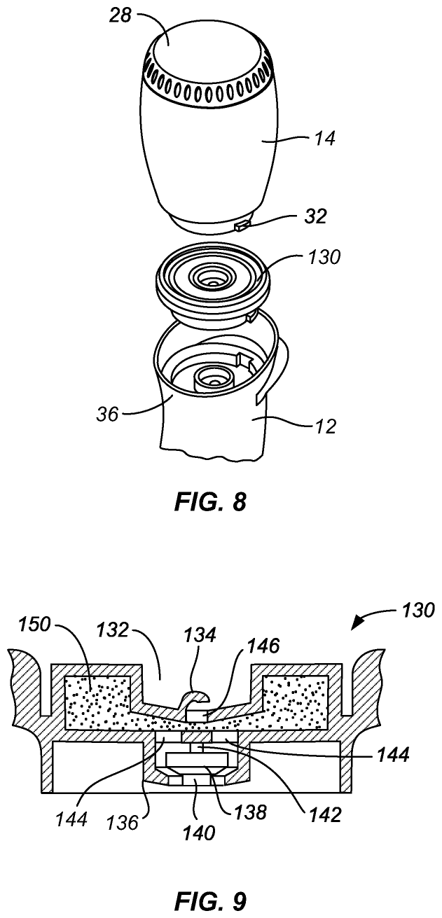

Referring now to FIGS. 8 and 9, a material release cartridge 130 may be provided, typically for mounting between the lower based 32 of the reservoir 14 and the upper end 36 of the main body 12, as seen in FIG. 8.

As seen in FIG. 9, the material release cartridge 130 is intended to be “modular” in that it can attach to both the lower end 32 of the reservoir 14 and the upper end 36 of the main body 12 in a manner analogous to that shown for direct attachment as shown in FIGS. 7A and 7B. In particular, the material release cartridge 130 typically has an inlet port 132 with a displacement element 134 positioned to engage and open the one-way valve 36 in the bottom of the reservoir 14. Similarly, the material release cartridge 130 has a one-way valve structure 136 on its lower surface. The one-way valve includes a valve plug 138 and a spring attachment 142 which allow the valve plug to be raised when engaging the displacement element 114 of the upper body cap 60 of the main body 12. In this way, fluid from the reservoir 14 may flow through inlet 146 of the material release cartridge 130 where it enter the material chamber 150 where it can dissolve, disperse, or otherwise release and combine a desired material with a fluid stream which then passes outwardly through outlet openings 144 and through valve port 140 into the main body from where it is pumped through the tubular nozzle member 116.

Referring now to FIG. 10, the personal hygiene device 10 of the present invention may be readily held in a single hand H of a user with, for example, a thumb T, depressing either the flow increase button 20 or flow decrease button 22. Similarly, an index finger F may be used to depress the on/off button 24 on the opposite side of the personal hygiene device (not visible in FIG. 10). The user could, of course, turn the device around so that the thumb is used to operate the on/off button 24 while one or two fingers F are used to operate the flow increase and decrease buttons 20 and 22. Generally, the user will hold the device in a vertical orientation, as illustrated, so that fluid flows by gravity from the reservoir 14 into the main body 12, as shown.

Referring now to FIG. 11, the personal hygiene device 10 of the present invention preferably has rechargeable batteries 76, as previously described. The batteries may be recharged in a recharging station 160 having a receptacle 62 for receiving the conical lower section 92 of the main body 12. A recharging element 64 will be aligned with the charging coil 94 in the main body 12 so that the batteries may be recharged. Optionally, an on/off switch 166 may be provided on the recharging station 160. Referring now to FIG. 12, the personal hygiene device 10 of the present invention may further include structure for disinfecting fluid while the fluid is maintained within the reservoir 14. For example, one or more ultraviolet light sources, such as ultraviolet light emitting diodes 170 may be included in an upper surface of the upper body cap 60. By providing a reservoir having a bottom which is transparent to ultraviolet energy, the LED's may irradiate the fluid with ultraviolet energy in order to disinfect the fluid therein.

While the present invention has been described herein with respect to certain illustrated embodiments, those of ordinary skill in the art will recognize and appreciate that it is not so limited. Rather, many additions, deletions, and modifications to the illustrated embodiments may be made without departing from the scope of the invention as claimed, including legal equivalents thereof. In addition, features from one embodiment may be combined with features of another embodiment while still being encompassed within the scope of the invention as contemplated by the inventors. Further, embodiments of the disclosure have utility with different and various tool types and configurations.

What is claimed is:

1. A hand-held personal hygiene device comprising:
a main body having an upper end with an inlet port and a lower end with an outlet port;
a pump within the main body, wherein the pump is configured to pump a fluid from the inlet port to an outlet port at the lower end;
a motor in the main body connected to drive the pump;
a reservoir having a lower base configured to removably seat in the upper end of the main body and a self-closing valve that engages the inlet port of the main body to open and allow fluid in the reservoir to flow to the pump when the lower base is seated in the upper end of the main body; and
a tubular nozzle member having a lower end comprising one or more nozzle outlets and an upper end configured to be removably secured to the outlet port of the main body, wherein fluid pumped by the pump in the main body will flow through the tubular nozzle member to the one or more nozzle outlets.

2. The hand-held personal hygiene device as in claim 1 wherein the reservoir and the main body are aligned to form a liquid flow path that may be held vertically to allow gravity flow from the reservoir to the pump

3. The hand-held personal hygiene device as in claim 1 wherein the main body has an upper cylindrical section to fit a user's hand and a lower conical section to connect to the tubular nozzle member.

4. The hand-held personal hygiene device as in claim 3 wherein the upper cylindrical section and the lower conical section each have a longitudinal axis and wherein the axis of the lower conical section is oriented at an angle in a range from 10° to 30° relative to the axis of the upper cylindrical section to deflect attachment of the tubular nozzle member.

5. The hand-held personal hygiene device as in claim 3 wherein the motor is controlled by an on/off button, a flow increase button, and a flow decrease button.

6. The hand-held personal hygiene device as in claim 5 wherein the on/off button is on a first side of the cylindrical section of the main body so that the user can operate the on/off button with one of either a thumb or finger(s) and the flow increase and the flow decrease buttons are on an opposite side of the of the cylindrical section of the main body so that the user can operate the flow increase and the flow decrease buttons with the other of either the thumb or the finger(s).

7. The hand-held personal hygiene device as in claim 1 wherein the reservoir has at least one of a screw attachment, a press fit attachment and a latched attachment with the upper end of the main body.

8. The hand-held personal hygiene device as in claim 1 wherein the self-closing valve of the reservoir comprises a spring-loaded valve that is configured to be displaced upwardly to open.

9. The hand-held personal hygiene device as in claim 8 wherein the inlet port of the main body comprises a displacement element positioned to displace a valve plug of the self-closing outlet valve of the reservoir upwardly to open said self-closing valve when the reservoir is attached to the main body.

10. The hand-held personal hygiene device as in claim 3 further comprising a pair of latching members at the upper end of the tubular nozzle member, wherein said latching members engage locking surfaces in an open lower end of the main body when the upper end of the tubular nozzle member is inserted into the open lower end of the main body.

11. The hand-held personal hygiene device as in claim 10 further comprising release buttons disposed through a wall the lower conical section of the of the main body and configured to be pushed inwardly to disengage the latching members from the locking surfaces.

12. The hand-held personal hygiene device as in claim 11 wherein the release buttons are disposed on opposites side of the of the conical section of the main body so that the user can squeeze the release buttons between a thumb and a finger to disengage the tubular nozzle member from the conical section of the main body.

13. The hand-held personal hygiene device as in claim 1 further comprising a material release cartridge configured to be inserted between the lower end of the reservoir and the upper end of the main body.

14. The hand-held personal hygiene device as in claim 13 wherein the material release cartridge has an interior volume filled with a soluble or dispersible material, an upper end with an inlet port configured to mate with and open the self-closing valve in the bottom of the reservoir, and a lower end with a self-closing valve configured to be opened by the inlet port of the main body when the material release cartridge is seated in the upper end of the main body.

15. The hand-held personal hygiene device as in claim 1 further comprising disinfecting energy structure in the inlet end of the main body.

16. The hand-held personal hygiene device as in claim 15 wherein the disinfecting energy structure comprises one or more light emitting diodes configured to direct ultraviolet energy into an interior of the reservoir when the reservoir is seated in the upper end of the main body.
